(19) United States
(12) Patent Application Publication
(10) Pub. No.: US 2024/0082058 A1
(43) Pub. Date: Mar. 14, 2024

(54) SURGICAL INSTRUMENT WITH GUARDED BLADE

(71) Applicant: OPTICGON LLC, Flushing, NY (US)

(72) Inventor: Ian CHAN, Flushing, NY (US)

(21) Appl. No.: 18/501,879

(22) Filed: Nov. 3, 2023

Related U.S. Application Data
(63) Continuation-in-part of application No. 17/385,580, filed on Jul. 26, 2021.
(60) Provisional application No. 63/056,882, filed on Jul. 27, 2020.

(51) Int. Cl. A61F 9/007; A61B 90/00

(52) U.S. Cl. CPC A61F 9/00736; A61B 90/03; A61B 2017/32113

(57) ABSTRACT

This disclosure relates to a surgical instrument assembly useful during retinal surgery. In particular, a surgical instrument described herein may be constructed from a small gauge. A guarded blade having a reversibly exposed sharp blade tip is described. A sleeve may be used to guard the blade tip while leaving a sharp cutting tip exposed. Use of a sleeve to guard the blade while allowing the sharp tip to remain exposed may allow for safer dissection of tissues. When used for surgeries involving the eye, a blade having a polymer sleeve and a reversibly exposed tip may inhibit and/or avoid the occurrence of inadvertent retinal tissue injury. The tip can be covered with a movable cylindrical shield to protect the tip during inserting into the eye then retracted to expose the tip during the cutting procedure. For example, using a small gauge blade guarded by a polymer sleeve during retinal surgical procedures may reduce or inhibit injuries to the retinal tissue when used during retinal surgical procedures such as membrane peeling and/or dissection. This disclosure relates to a surgical instrument, in particular a vitreoretinal surgical assembly, having a guarded blade useful during retinal surgery. For example, a vitreoretinal surgical assembly may be used to dissect at least one of adherent internal limiting membrane, epiretinal membrane, diabetic tractional membranes and/or proliferative vitreoretinal disorders.

### RELATED APPLICATION

This continuation-in-part application claims the benefit of priority to U.S. application Ser. No. 17/385,580 filed Jul. 26, 2021, which claims priority to U.S. Provisional Application 63/056,882 filed Jul. 27, 2020, the contents of each of which are herein incorporated in its entirety.

### FIELD

This disclosure relates to a surgical instrument that includes a structurally guarded blade which is useful during retinal surgery. The structurally guarded blade can be further protected during trocar traversal. The surgical instrument may be used to treat macula diseases involving membranectomy and proliferative vitreoretinal disorders by dissecting membranes from a retina. For example, membranes such as adherent internal limiting membrane (ILM), epiretinal membrane (ERM), and/or diabetic tractional membranes may be dissected from a retina using the surgical instrument having a guarded blade and described herein.

### BACKGROUND

The following includes information that may be useful in understanding this disclosure. It is not an admission that any of the information, publications or documents specifically or implicitly referenced herein is prior art, or essential, to the presently described or claimed subject matter. All publications and patents mentioned herein are hereby incorporated herein by reference in their entirety.

A challenge to surgeons, in particular vitreoretinal surgeons, is the separation of adherent internal limiting membranes, epiretinal membranes, diabetic tractional membranes, and/or proliferative membranes from the neurosensory retina without injury and/or harm during surgery. The removal of these membranes is required in a wide variety of pathological conditions and surgical situations.

Various intraocular picks, scrapers and forceps have previously been described for use in vitreoretinal surgeries (Kuhn F et al. Th Tano Diamond Dusted Membrane Scraper. Indications and contraindications. Acta Ophthalmol Scand. 1998 December; 76 (6):754-5; Inoue M et al., 25-Gauge Cannula System with Microvitreoretinal Blade Trocar, Am J Ophthalmol. 2007 August; 144(2):302-4; Inoue M et al. Comparison of the effects of 23-gauge and 25-gauge microincision vitrectomy blade designs on incision architecture. Clin Ophthalmol. 2014 Nov. 19; 8:2307-18; Randolph L. et al. Modification of the Tano Diamond Dusted Scraper. Retina. 2000; 20(4):427). However, there is a significant risk of causing retinal damage when using most of the known intraocular picks, scrapers, and/or forceps to dissect these membranes, for example, if too much force is applied. In addition, at times these instruments fail to sufficiently remove membranes, in particular proliferative membranes, resulting in recurrence and/or inadequate treatment of these membranes.

All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Citation or identification of any document in this application is not an admission that such document is available as prior art.

### SUMMARY

The embodiments described herein have many attributes and aspects including, but not limited to, those set forth or described or referenced in this Summary. It is not intended to be all-inclusive and the embodiments described herein are not limited to or by the features or embodiments identified in this Summary, which is included for purposes of illustration only and not restriction.

This disclosure provides for a surgical blade which can force-regulate to limit the penetration depth of a blade when performing vitroretinal surgeries. In some aspects, the sharp edge of a surgical blade creates a flap in the ocular membrane while a polymer overcoating guard prevents damage to the deeper retinal tissue. The polymer-guard will also act like the guard of a razor blade for shaving, which helps create a proper amount of retinal indentation without entering too deep into retinal tissue and aid in the flap creation. The polymer-guard will also regulate the feedback force an operator manually receives when penetrating beyond a preselected penetration depth to limit penetration of a cutting instrument beyond a preselected penetration depth during ophthalmic surgery.

In some aspects, this disclosure provides for an ophthalmic treatment tool for use in an ophthalmic surgery, such as a surgical instrument for use in an ophthalmic surgery may include a grip portion, a rod-shaped body attached to one end of the grip portion, and/or a pointed blade that tapers from the other end of the rod-shaped body. A portion of a blade may be angled with respect to a proximate section of the surgical instrument such that an angle between the blade portion and the proximate section may be in a range from about 35 degrees to about 135 degrees. In some aspects, the blade comprises a tip section and a proximate section positioned such that an angle between the tip portion and the proximate section is about 90 degrees. In some aspects, the blade comprises a shape-memory metal. In some aspects, the blade can be configured to have a first angle and a second angle, wherein the first angle is present when the blade is deformed and the second angle is present when the blade is not deformed. In some aspects, the blade can be configured to have a 180 degree angle during trocar insertion but return to its native angle configuration upon passing through the trocar and entering the eye. In some aspects, the native angle configuration is about 90 degrees.

In some aspects, a sleeve such as a flexible polymer guard may encompass at least part of the sharp portion of the blade such that a predetermined length of a tip portion is exposed.

A sleeve, such as a flexible polymer guard may coat a portion of a blade. In various aspects, an amount of exposed tip of the blade may vary depending on use. In some aspects, the sleeve can comprise a drug. The drug can be impregnated within the polymer, by a similar principle of a drug-eluting stent. In some aspects, the drug is an anesthetic, antibiotic, antimicrobial, anti-VEGF, or wound healing drug. In some aspects, the wound healing drug is an anti-inflammatory drug. In some aspects, the drug is selected from: bevacizumab (Avastin), ranibizumab (Lucentis), aflibercept (Eylea), Vabysmo (faricimab), Ranibizumab-nuna (Byooviz), Ranibizumab-eqrn (Cimerli), Brolucizumab (Beovu), Faricimab (Vabysmo), Aflibercept (Eylea HD), Pegcetacoplan (Syfovre), Avacincaptad pegol (Izervay), Triamcinolone acetonide, Ganciclovir, Clindamycin, Foscarnet, Fomivirsen Methotrexate, Vancomycin, Ceftazidime, Amikacin, Amphotericin B, Voriconazole, and Dexamethasone. The drug-impregnated polymer, and sleeve formation, can be prepared using methods similar to those of preparing drug eluting vascular stents and are described in U.S. Pat. Nos. 8,257,724 and 9,358,096.

During use a polymer edge of a sleeve may cause a slight indentation of the retina and pushing the retina into microscopic folds, thus creating a safe dissection plane for the blade during flap creation. In some aspects, the drug impregnated within the sleeve is released into the retina upon contact of the sleeve portion with the retina, resulting in transfer of the drug to the selected portion of the eye. The eluted drug can thereby enhance post-operative recovery, or further treat a retinal disease in the subject.

The polymer guard can also serve as a stopper to prevent over-penetration of the blade into neurosensory retina. The polymer guard serves the function similar to the skin guard of a razor, which creates a mild indentation into the retina and guides the blade to lift the superficial proliferative membranes or anatomical membranes which can include or exclude the internal limiting membrane (ILM).

The polymer can be made of a heat-shrinking material that is safe for medical use. The polymer can be made of, but not limited to, medical grade fluoropolymer, fluoroelastomer, polyolefin, polyvinylidene fluoride, silicone, silicone rubber, polyethylene, polyester, polyester block amide (PEBA) and other materials suitable for heat shrink tubing. In one embodiment, the polymer can be a heat-shrinking material which can be inserted to cover the blade and exposing the tip, thus achieving a tight fit over the contour of the blade.

The thickness of the polymer sleeve can be selected or adjusted to create different guard thickness, which would be suitable for different types of retinal membranes i.e. internal limiting membrane which requires less indentation vs a tractional epiretinal membrane, which requires a greater degree of indentation and therefore, a thicker guard sleeve.

In some aspects, this disclosure provides for a vitreoretinal surgical instrument, for example, a vitreoretinal surgical instrument may include a handheld small diameter blade extending from a base unit, having a length and a diameter equal to or smaller than 20 gauge, a support sleeve located around an end of the vitrectomy instrument portion adjacent to the base unit, and extending from the base unit to a location along the length of the vitreoretinal surgical instrument portion, wherein a distal end of the vitreoretinal surgical instrument portion is configured to enter an eye, while the support sleeve is configured to remain outside a surface of an eye when in use, wherein the support sleeve includes a rod.

A vitreoretinal surgical instrument assembly may include a blade of about 23 gauge, about 25 gauge, or about 27 gauge. A surgical instrument such as a vitrectomy instrument may include a stainless steel or titanium rod. In some aspects, the surgical instrument may comprise a material having pseudoelastic properties. In some aspects, the material having pseudoelastic properties is a shape memory alloy. In some aspects, the shape memory alloy can include or exclude nitinol. In some aspects, the nitinol comprises about 55% nickel and about 45% titanium.

In some aspects, a vitreoretinal surgical instrument may include a rod that tapers to a sharp blade structure at a first end.

A vitreoretinal surgical instrument assembly may include a tip on a blade angled at about 45 degrees to about 90 degrees. In some aspects, a shaft of the instrument may be positioned such that an angle between the shaft and the proximate section of the instrument may be angled at about 0 degrees and about 15 degrees to enable an improved angle of approach to the retina.

A vitreoretinal surgical instrument assembly may include a tip of a blade exposed by varying amounts, from about 0.25 mm to about 2 mm. The rest of the blade may be covered by a semi-rigid polymer guard depending on the type of membranes that need to be peeled or dissected.

A sleeve, a polymer guard, may include a tip of the guard that is made of heat-shrinking polymer. In some aspects, a blade may be inserted into a polymer guard and may further be secured to the blade with medical grade epoxy. The sleeve acts as a coating on a portion of the blade.

Sleeves may be formed from medical grade materials including but not limited to heat-shrinking material such as a fluoropolymer, in particular, a medical grade fluoropolymer, polymers such as fluoroelastomer, polyolefin, polyvinylidene fluoride, polyethylene, polyester, polyester block amide (PEBA), silicone, silicone rubber, ceramics, and/or other medical grade materials known in the art. Materials for use in sleeves may be selected for particular property, for example, a predefined deflection property, a tensile modulus of elasticity, tensile elongation, flexural strength, flexural modulus of elasticity, example made of heat shrinking polymer material.

In one embodiment, a polymer for use in a sleeve may be a heat-shrinking material which may cover the blade and expose a cutting section and/or a portion of a tip section, thus achieving a tight fit over the contour of the blade. A sleeve, such as a polymer guard, may have a thickness of the polymer guard in a range from about 0.1 mm to about 1 mm.

In some aspects, this disclosure provides for a surgical handpiece comprising:
a vitreoretinal surgical instrument as described herein,
a longitudinally movable shield;
a rod-shaped body which includes a control slider;
wherein the control slider is configured to be movable along the longitudinal axis of the rod-shaped body;
wherein the control slider is configured to be positioned laterally on the rod-shaped body and is configured to control the longitudinal movement of the shield, and
wherein the longitudinally movable shield is configured to have a first position where the shield is extended to cover the blade and a second position where the shield is retracted to expose the blade.
   In some aspects, the vitreoretinal surgical instrument comprises:
a blade having a diameter greater than about 34 gauge comprising:
   a cutting surface positioned at a first end of the blade and configured to cut an eye tissue;
a sleeve positioned proximate at least a portion of the blade and configured to displace at least some tissue proximate a predetermined cut location during use and further to control a depth to which the cutting surface of the blade enters tissue at the predetermined cut location during use and wherein the sleeve is positioned such that the sleeve substantially remains outside the surface of the eye during use;
a shaft coupled to blade; and
wherein the sleeve surrounds at least a portion of the blade.

In some aspects, the control slider comprises a protrusion configured to be manipulated by a digit of an operator. In some aspects, the digit is the forefinger or thumb.

The longitudinally movable shield surrounds the blade when in the first position.

In some aspects, the longitudinally movable shield is a cylinder. In some aspects, the cylinder is an circular cylinder or an oval cylinder.

In some aspects, this disclosure provides for a kit comprising a surgical handpiece as described herein and a trocar. In some aspects, the surgical handpiece comprises:
a vitreoretinal surgical instrument as described herein,
a longitudinally movable shield;
a rod-shaped body which includes a control slider;
wherein the control slider is configured to be movable along the longitudinal axis of the rod-shaped body;
wherein the control slider is configured to be positioned laterally on the rod-shaped body and is configured to control the longitudinal movement of the shield, and
wherein the longitudinally movable shield is configured to have a first position where the shield is extended to cover the blade and a second position where the shield is retracted to expose the blade.

In some aspects, this disclosure provides for a method of presenting a cutting blade to the vitreous humor of the eye of a subject, the method comprising:
inserting a trocar into the eye of a subject;
inserting a vitreoretinal surgical instrument comprising a blade through the trocar wherein the blade is surrounded by a longitudinally movable shield; and
retracting the longitudinally movable shield to expose the blade.
In some aspects, the cutting blade is protected during trocar insertion such that the blade is not susceptible to bend or breakage during trocar insertion. In some aspects, the vitreoretinal surgical instrument is a vitreoretinal surgical instrument as described herein.

It is an object if the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The drawings form part of the present specification and are included to further demonstrate certain aspects of the embodiments described herein. The following detailed description, given by way of example, may best be understood in conjunction with the accompanying drawings.

FIG. 1 depicts a top side perspective view of an embodiment of a small gauge surgical blade with a sleeve.

FIG. 2 depicts a bottom perspective view of an embodiment of a small gauge surgical blade with a sleeve.

FIG. 3 depicts a top side perspective view of an embodiment of a small gauge surgical blade with a sleeve.

FIG. 4 depicts a front perspective view of an embodiment of a small gauge surgical blade encompassed in a sleeve for use in a surgical instrument.

FIG. 5 depicts a top perspective view of an embodiment of a small gauge surgical blade encompassed in a sleeve for use in a surgical instrument.

FIG. 6 depicts a side view of an embodiment of a small gauge surgical blade encompassed in a sleeve for use in a surgical instrument.

FIG. 7 shows a magnified image of an end of a surgical instrument that includes a sharp tip of a surgical instrument extending beyond the sleeve.

FIG. 8 shows a magnified image of a side perspective view of an end of a surgical instrument that includes a sharp tip of a surgical instrument extending beyond the sleeve.

FIG. 9 shows a magnified image of a side perspective view of an end of a surgical instrument that includes a sharp tip of a surgical instrument extending beyond the sleeve.

FIG. 10A shows a representative embodiment of a handpiece comprising a small gauge surgical blade with a longitudinally movable protective shield extended to cover the surgical blade.

FIG. 10B shows a representative embodiment of a handpiece comprising a small gauge surgical blade with a longitudinally movable protective shield retracted to expose the surgical blade.

FIG. 11 shows a handpiece with a longitudinally moveable protective shield extended to cover the surgical blade while traversing through a trocar which crosses the sclera of an eye.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

As used herein, the term “about” means+/−10% of any measurable value. For example, the term “about 10 millimeters” refers to a range of 9 to 11 millimeters.

When treating disorders of the retina, surgeons are often required to separate membranes due to a wide variety of pathological conditions and surgical situations. When surgeons dissect these membranes from the patient's retina, they attempt to do so in a manner that reduces and/or inhibits injury and/or harm to the retina. A preferred embodiment of a surgical instrument used to dissect these membranes and described herein is constructed such that it reduces and/or inhibits injury and/or harm to the patient's retina.

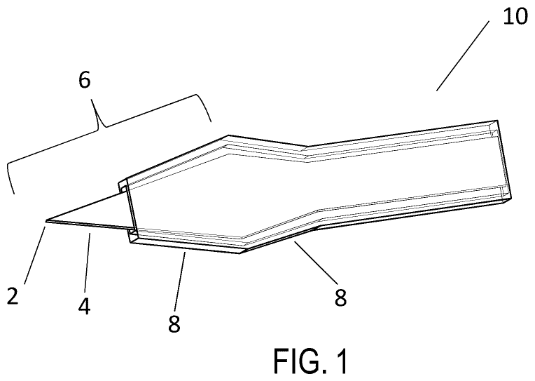

Membranes such as adherent internal limiting membranes, epiretinal membranes, diabetic tractional membranes, and/or proliferative membranes may be dissected from the neurosensory retina using a surgical instrument having a guarded blade as described herein and shown in FIG. 1.

As depicted in FIG. 1, a substantially planar surgical instrument 10 includes cutting surface 2 having exposed tip 4 of blade 6. Blade 6 may include a portion that is surrounded by sleeve 8.

The blade may be constructed from metal. Metals may be selected for a particular material property such as a predetermined spring force constant, hardness, brittleness, ductility, toughness, strength, rigidity, and/or any properties of interest known in the art. Metals may include but are not limited to medical grade metals, stainless steel such as grades 304, 316, or 316L, alloys such as cobalt-chromium alloys or titanium alloys, titanium and/or combinations thereof.

Blade thickness may vary in a range from about 34 gauge (i.e., 0.16 mm) to about 10 gauge (i.e., 2.6 mm). In particular some embodiments may include a blade having a thickness in a range from about 30 gauge (i.e., 0.26 mm) to about 20 gauge (i.e., 0.81 mm). For example, a preferred embodiment may have a blade having a thickness in a range from about 28 gauge (i.e., 0.32 mm) to about 22 gauge (i.e., 0.64 mm).

Sleeve 8 acts as a guard for blade 6. In some embodiments, a sleeve may serve as a distance restrictor. For example, a sleeve may be used to control a depth within a tissue to which a blade may enter. In particular, a sleeve may inhibit and/or prevent a blade from entering too deeply into the tissue.

In some embodiments of a vitreoretinal surgical assembly, a sleeve on a surgical instrument may cause at least a portion of the tissue to deflect, displace, and/or indent proximate an area of tissue to be cut and/or dissected during use.

For example, a sleeve may displace and/or indent at least a portion of retinal tissue and thus create a tangential plane for a cutting surface of a blade to cut. A thickness of the sleeve may be selected and/or adjusted based on the type of tissues to be cut and/or dissected. Different types of retinal membranes may benefit from differing amounts of displacement to be cut or be dissected. For example, an internal limiting membrane may require less indentation of retinal tissue for cutting and/or dissection than a tractional epiretinal membrane. In particular, when cutting and/or dissecting a tractional epiretinal membrane a greater degree of indentation of the retinal tissue may be desired, and therefore a thicker sleeve on the surgical instrument may be used as a guard.

In some embodiments, a sleeve may have a predetermined thickness. A sleeve thickness may be in a range from about 0.01 mm to about 2 mm. For example, a sleeve thickness for an embodiment of a surgical instrument may be in a range from about 0.05 mm to about 0.8 mm. In particular, a sleeve for a surgical instrument may be in range from about 0.1 mm to about 0.5 mm. A sleeve may include a single layer of material. In some embodiments, a sleeve may have multiple layers of the same or differing materials.

Sleeves may be formed from medical grade materials including but not limited to heat-shrinking material such as a fluoropolymer, in particular, a medical grade fluoropolymer, polymers such as fluoroelastomer, polyolefin, polyvinylidene fluoride, polyethylene, polyester, polyester block amide (PEBA), silicone, silicone rubber, ceramics, and/or other medical grade materials known in the art. Materials for use in sleeves may be selected for particular property, for example, a predefined deflection property, a tensile modulus of elasticity, tensile elongation, flexural strength, flexural modulus of elasticity, example made of heat shrinking polymer material.

In one embodiment, a polymer for use in a sleeve may be a heat-shrinking material which may cover the blade and expose a cutting section and/or a portion of a tip section, thus achieving a tight fit over the contour of the blade.

Sleeve materials may be positioned over at least a portion of a surgical instrument without the use of adhesive in some instances. For example, sleeve materials may be positioned over a portion of a blade without an adhesive. In an embodiment, sleeve materials may be positioned over a portion of a blade and a portion of the shaft without the use of adhesive.

In some embodiments, heat-shrinking material made be used in a sleeve such that the sleeve may achieve a tight fit over a contour of the blade to act as a guard. In particular, a medical grade fluoropolymer may be used to achieve a tight fit over a blade.

A sleeve may be formed from a coating such as a polymer coating. Polymers for use in sleeves may be selected for particular property, for example, a predetermined deflection property, a tensile modulus of elasticity, tensile elongation, flexural strength, flexural modulus of elasticity, and/or a combination of predetermined properties.

A sleeve may be formed on a blade by dip coating, deposition, injection molding such as liquid silicone rubber (LSR) injection molding, 3D printing such as fused deposition, digital light synthesis, for example, Continuous Liquid Interface Production™ (CLIP™ by Carbon), inserting the blade through a stretchable and/or heat-shinkable polymer sheath, laser cutting or a combination of methods. For example, a guarded blade may be formed by coating a blade with a coating and then removing a portion of the coating. In particular, a guarded blade may be formed by coating a blade with a polymer coating and then selectively removing a portion of the coating, for example, using a laser removal process. The laser removal process can include or exclude presenting a UV-wavelength laser. In some embodiments, UV-emitting (355-nm) neodymium:solid-state lasers, such as the Avia laser from Coherent, can be used to cut a wide range of plastics, including PE, PP, ABS, and PBT, using controlled exposure and/or intensities, as determined by the user for the appropriate plastic by methods known in the art.

In an embodiment, a surgical instrument may be formed by dip coating a blade to form the sleeve. When formed in this manner the sleeve may surround the blade. In some embodiments, multiple dips are needed to form a sleeve having the desired thickness. For example, dip coating some materials may result in a sleeve layer having a thickness less than a predetermined thickness. Thus, a surgical instrument may be dipped multiple times to achieve the predetermined sleeve thickness. In some embodiments, a thickness of a dip coating may be controlled by formulation of the material. In particular, if a silicone dispersion is used to form the sleeve a wall thickness of the sleeve may be controlled by adjusting the percent solids concentration in the silicone dispersion. Further, in some embodiments a sleeve thickness may be controlled when dip coating by varying the number of dips and/or adjusting the percent solids concentration of the silicone dispersion. Materials used for dip coating may include silicone dispersions, medical grade epoxy, material fluorobond, etc. In some embodiments, the dip solution comprises a melted polymer, or a polymer dissolved in a volatile solvent. When the polymer is dissolved in a volatile solvent, when the blade is immersed in the dipping solution then extracted from the dipping solution, the volatile solvent is allowed to evaporate for a preselected amount of time after the blade is extruded from the dipping solution. In some embodiments, the rate of entry and/or exit of the blade from the dipping solution is controlled to maintain about general uniformity of thickness over a portion of the blade.

In some embodiments, a sleeve may be formed on a blade by inserting a tubing of a heat-shrink material having a diameter larger than that of the blade over all or a portion of the blade, followed by presenting UV light or heat to the heat-shrink tubing to cause the heat-shrink tubing to shrink to conform to about the physical configurations of the blade. In some embodiments, the heat-shrink tubing can be cut after shrinking the material with a laser or knife to expose a portion of the blade as described herein.

In some embodiments, the blade may be rigid. A portion of the blade may be angled. In some embodiments, a surgical instrument may have a shaft. In some instances, at least a portion of the shaft may be flexible. For example, a surgical instrument may include a shaft having at least a portion made of silicone.

In particular, a guarded blade is useful during retinal surgery when dissecting membranes from a retina. In particular, the surgical instrument may be used to treat proliferative vitreoretinal disorders by dissecting membranes from a retina. The membranes which the guarded blades described herein can be used on can include or exclude: adherent internal limiting membrane (ILM), epiretinal membrane (ERM), fibrovascular membranes from proliferative vitreoretinopathy, and/or diabetic tractional membranes, all of which are in or about the retina.

A surgical instrument described herein and may be used in ophthalmic surgeries, in particular, vitrectomy. In some embodiments, a vitrectomy surgical instrument may include a rod-shaped body having a blade positioned proximate a first end of the body. The blade may include a cutting surface at a tip of the blade.

This disclosure relates to a polymer-guarded blade and in more detail, to a new instrument which is useful during retinal surgery for dissecting adherent internal limiting membrane, epiretinal membrane, diabetic tractional membranes and proliferative vitreoretinal disorders.

An edge of a sleeve, for example, a polymer guard may allow for slight indentation of a retina during use of a surgical instrument. Further, the surgical instrument may push the retina into microscopic folds during use, thus creating a safe dissection plane for the blade during flap creation. The sleeve may act as a polymer guard and/or serve as a stopper to inhibit and/or prevent over-penetration of the blade into neurosensory retina. The polymer guard may cause a mild indentation into the retina and guide the blade to lift the superficial proliferative membranes.

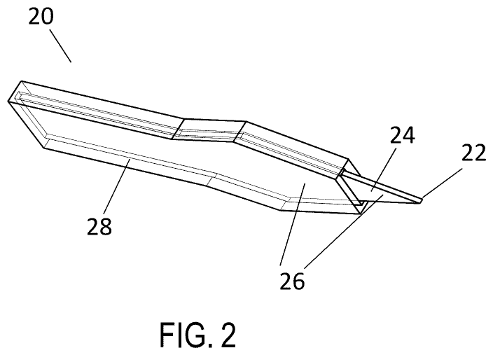

As shown in FIG. 2, surgical instrument 20 may be substantially planar. Surgical instrument 20 has a cutting surface 22 on tip section 24 of blade 26. Sleeve 28 surrounds at least a portion of blade 26.

A sleeve, for example, a flexible polymer guard may coat a portion of the blade in a manner that allows a cutting surface of the blade to be exposed. During an ophthalmic procedure, such as vitrectomy, a polymer guard may allow for a slight indentation of the retina, pushing the retina into microscopic folds, and thus create a safe dissection plane for the blade during flap creation. Further, a sleeve or polymer guard may serve as a stopper to prevent over-penetration of the blade into neurosensory retina and further create a mild indentation into the retina and guide the blade to lift the superficial proliferative membranes. The sleeve, for example, a polymer guard may be made of a heat-shrinking material safe for medical use medical grade fluoropolymer, fluoroelastomer, polyolefin, polyvinylidene fluoride, silicone, silicone rubber, polyethylene, polyester, polyester block amide (PEBA) and other materials suitable for heat shrink tubing when the polymer is a heat-shrinking material the polymer guard may achieve a tight fit over the contour of the blade.

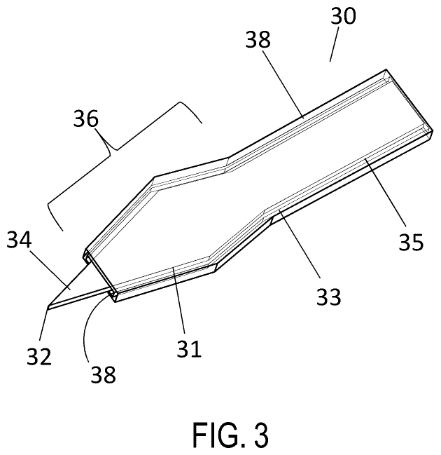

FIG. 3 depicts surgical instrument 30 having cutting surface 32 on tip section 34 of blade 36. Sleeve 38 surrounds at least a portion of blade 36 and at least a portion of shaft 35. A shaft may be made from a rigid material such as stainless steel, titanium, alloys, or combinations thereof, flexible materials such as silicone, and/or combinations thereof. In some embodiments, a shaft may have one or more sections. For example, a shaft may have one or more flexible portions made of silicone.

In some embodiments, a sleeve may include a thickness of the polymer sleeve selected and/or adjusted to create guard of a predetermined thickness, which would be suitable for different types of retinal membranes, for example, an internal limiting membrane requires less indentation than a tractional epiretinal membrane requires a greater degree of indentation and therefore, a thicker guard sleeve.

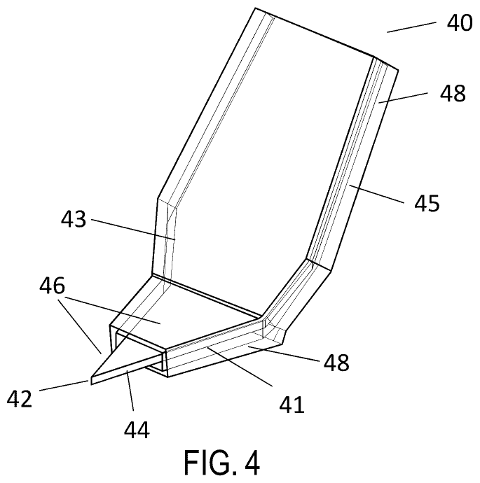

Surgical instruments may have one or more angled sections. As depicted in FIG. 4, in a small gauge surgical bent-blade 40, tip section 44 of blade 46 is exposed, thus not covered by the coating 48. Cutting surface 42 is positioned at an edge of tip section 44. Covered section 41 of blade 46 is angled with respect to angled section 43. In some instances, the tip section may be angled with respect to the blade such that an angle between the planes defining the tip section and an angled section of the blade is in a range between about 45 degrees to about 90 degrees. Further, FIG. 4 shows an angled section 43 that is angled with respect to shaft 45. For example, an angled section may be angled with respect to the shaft such that an angle between the planes defining the angled section of the blade and the shaft is in a range between about 0 degrees to about 15 degrees.

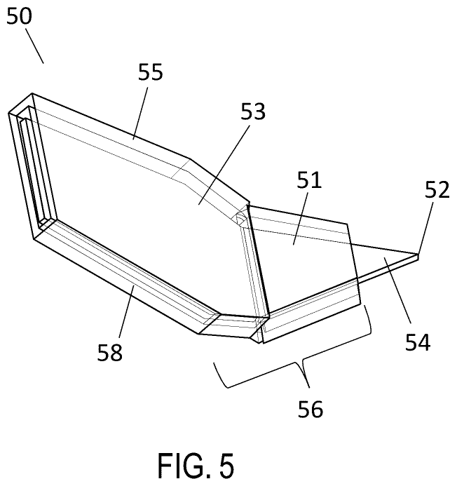

FIG. 5 illustrates an embodiment of a surgical instrument 50 having sections 51, 53 that positioned such that the sections are positioned at an angle to each other. Covered section 51 of blade 56 is angled with respect to angled section 53. In some instances, the tip section 54 may be angled with respect to the blade such that an angle between the planes defining the tip section and an angled section of the blade is in a range between about 45 degrees to about 90 degrees. Further, FIG. 5 shows an angled section 53 that is angled with respect to shaft 55. For example, an angled section may be angled with respect to shaft such that an angle between the planes defining the angled section of the blade and the shaft is in a range between about 0 degrees to about 15 degrees. As depicted in FIG. 5, tip section 54 of blade 56 is exposed. In some embodiments, a cutting surface 52 may be positioned at along one or more edges of the tip section.

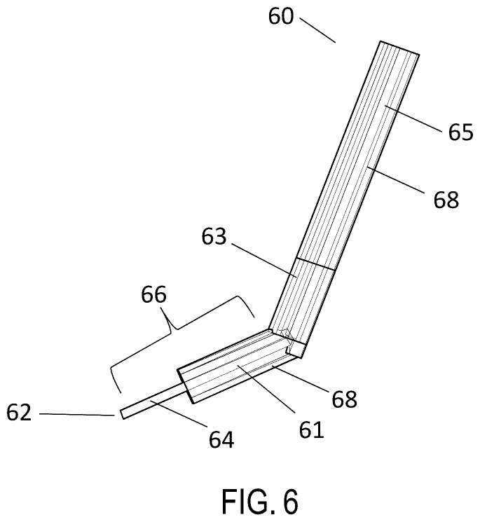

As shown in FIG. 6 a side view of surgical instrument 60 includes blade 66 having cutting surface 62 and sleeve 68. A blade may be rigid. Materials for use in blade and/or a geometry of the blade may be selected such that a predetermined property is achieved in the blade. As shown in FIG. 6, surgical instrument 60 and blade 66 may be angled. In some embodiments, tip section 64 of blade 66 may be positioned at an angle in a range between about 45° and about 90° degrees when measured from a plane extending through a center line of shaft 65. For example, as depicted in FIG. 6 tip section 64 and shaft 65 such that the combination forms a hockey-stick orientation. Sleeve may be formed from polymer. In some embodiments, a sleeve may surround at least in part a section of the blade 61. Alternatively, a sleeve may surround at least a section of the blade and/or shaft.

In some embodiments, surgical instruments may include a flexible portion, for example, a shaft having at least a portion made of silicone. In particular, a flexible shaft may bend, for example, when a blade touches the retina. Generally, a blade of the surgical instrument is generally rigid relative to the retina upon contact. A thickness of the metal in the blade may be in range from about 0.5 mm to about 1 mm.

In some embodiments, a surgical instrument for use in an ophthalmic surgery may include a grip portion, a rod-shaped body attached to one end of the grip portion, and/or a pointed blade that tapers from the other end of the rod-shaped body. A portion of a blade may be angled with respect to a proximate section of the surgical instrument such that an angle between the blade portion and the proximate section may be in a range from about 45 degrees to about 90 degrees. In some embodiments, a flexible polymer guard may encompass at least part of the sharp portion of the blade such that a predetermined length of a tip portion is exposed.

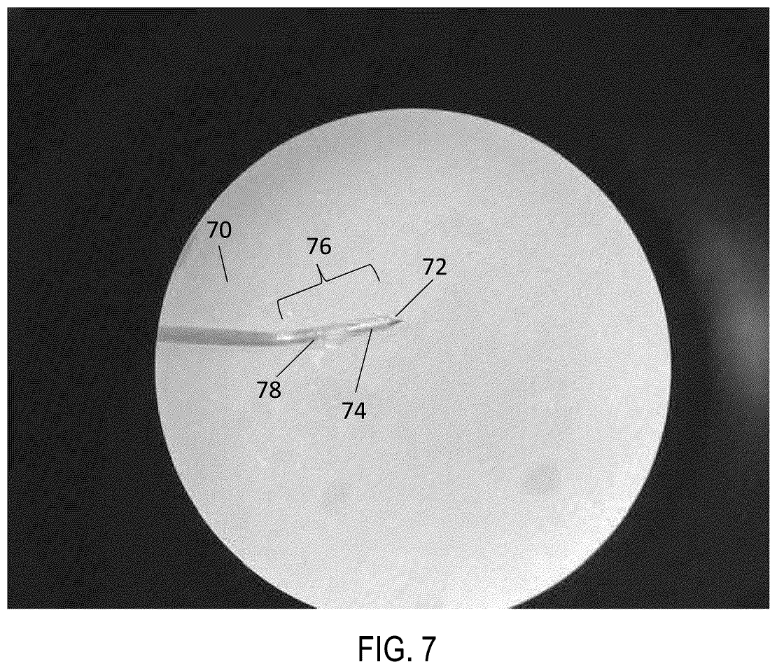

FIG. 7 shows a ten times magnified image of an end of a surgical instrument that includes a twenty-five gauge blade extending beyond a polymer sleeve. As shown the tip section of the blade is exposed. In some instances, a tip of the blade may be bent. As depicted in FIG. 7, surgical instrument 70 includes blade 76 having tip 74 and sleeve 78 surrounding at least a portion of blade 76. Tip section 74 may include angled cutting edge 72. In some instances, a tip section of a blade may have a predetermined thickness at the cutting surface.

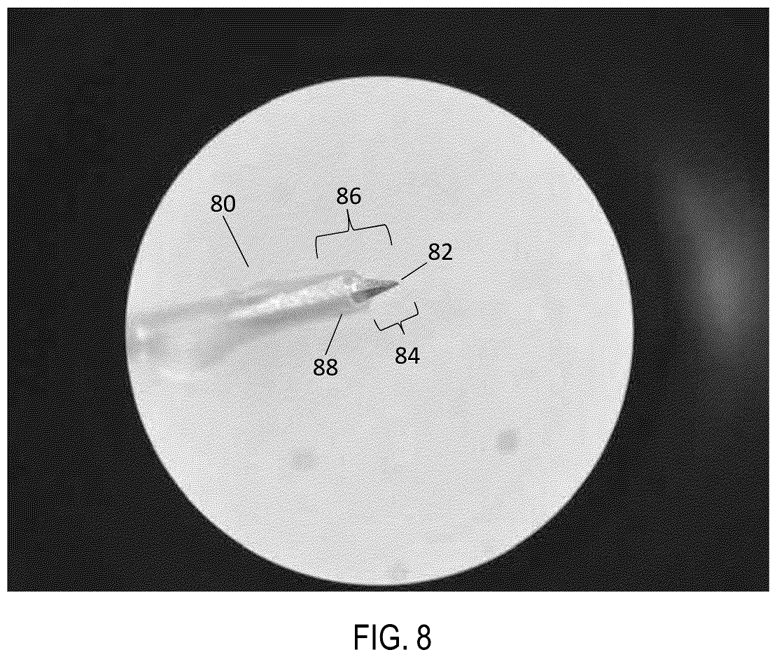

FIG. 8 shows an image of a side view of a surgical instrument magnified by thirty times. Surgical instrument 80 may include cutting surface 82 on tip section 84 of blade 86. Sleeve 88 covers at least a portion of blade 86. Cutting section may be angled. A blade may have a diameter less than about 1.0 mm. In some embodiments, a blade may have a diameter of less than about 0.81 mm. For example, a blade may have a diameter of less than about 0.51 mm. In an embodiment, a blade has a thickness in a range from about 0.5 mm to about 1 mm. In some embodiments, a blade for use in the surgical instrument described herein may have a diameter in a range from about 20 gauge to about 34 gauge. The gauge of metal diameter needle is inversely proportional to the diameter of said needle. For example, the diameter of a 34 gauge needle is 0.159 mm. The diameter of a 33 gauge needle is 0.21 mm. The diameter of a 32 gauge needle is 0.235 mm. The diameter of a 31 gauge needle is 0.261 mm. The diameter of a 30 gauge needle is 0.312 mm. The diameter of a 29 gauge needle is 0.337 mm. The diameter of a 28 gauge needle is 0.362 mm. The diameter of a 27 gauge needle is 0.413 mm. The diameter of a 26s gauge needle is 0.474 mm. The diameter of a 26 gauge needle is 0.464 mm. The diameter of a 25s gauge needle is 0.515 mm. The diameter of a 25 gauge needle is 0.515 mm. The diameter of a 24 gauge needle is 0.566 mm. The diameter of a 23s gauge needle is 0.642 mm. The diameter of a 23 gauge needle is 0.642 mm. The diameter of a 22s gauge needle is 0.718 mm. The diameter of a 22 gauge needle is 0.718 mm. The diameter of a 21 gauge needle is 0.819 mm. The diameter of a 20 gauge needle is 0.908 mm. The diameter of a 19 gauge needle is 1.067 mm. The diameter of a 18 gauge needle is 1.27 mm. The diameter of a 17 gauge needle is 1.473 mm The diameter of a 16 gauge needle is 1.651 mm. The diameter of a 15 gauge needle is 1.829 mm. The diameter of a 14 gauge needle is 2.109 mm. The diameter of a 13 gauge needle is 2.413 mm. The diameter of a 12 gauge needle is 2.769 mm. The diameter of a 10 gauge needle is 3.404 mm.

For example, in an embodiment, a twenty gauge blade may be covered at least in part by a polymer sleeve. For example, a surgical instrument may include a sharp tip section extending beyond the sleeve. The sharp tip section may be angled and rigid.

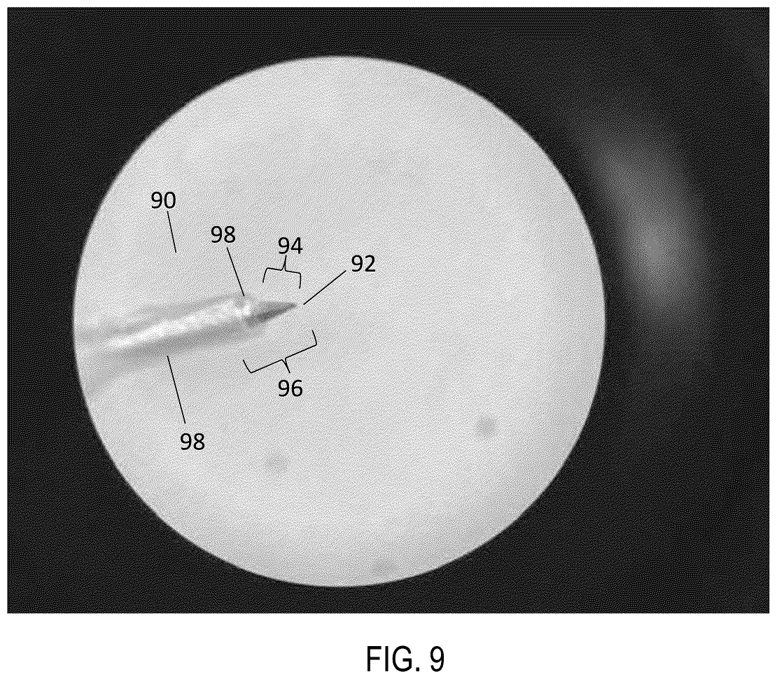

FIG. 9 shows a thirty times magnified image of a side perspective view of an end of a surgical instrument. As shown, blade 96 is twenty-five gauge. Sleeve 98 covers at least a portion of the surgical instrument, such as tip section 94. A predetermined portion of tip section 94 of surgical instrument 90 extends beyond sleeve 98.

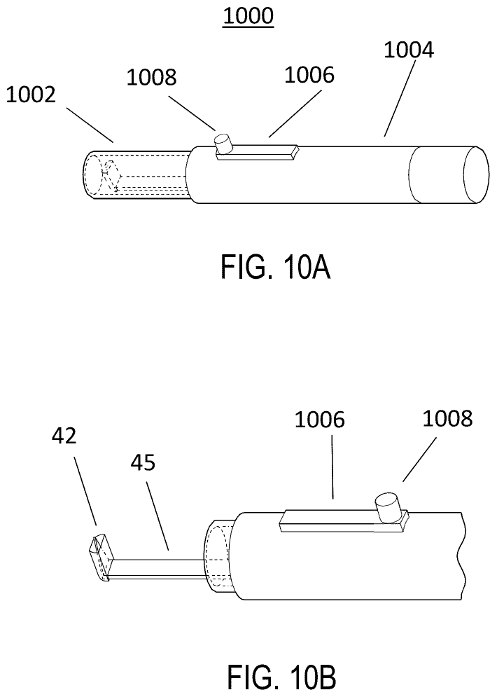

FIG. 10A shows an embodiment of a surgical handpiece 1000, comprising a rod-shaped body 1004, a longitudinally movable shield 1002 elongated to cover the cutting surface, a control slider 1006, and a protrusion 1008. FIG. 10B shows the surgical handpiece 1000 where the longitudinally movable shield 1002 is retracted so as to expose the cutting surface 42 and part of the shaft 45, when the vitreoretinal surgery device comprises two axes (e.g., it is bent).

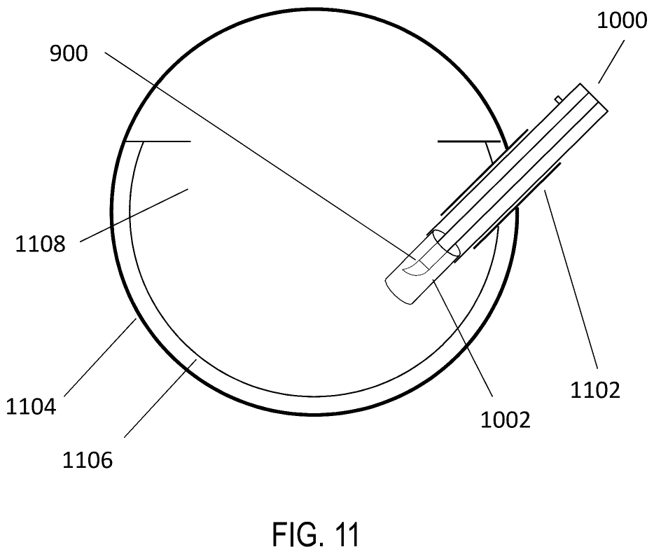

FIG. 11 shows the application of a representative surgical handpiece where the cutting blade portion 900 (which comprises a shaft and cutting surface, not explicitly marked) inserted through a trocar 1102. The trocar traverses the episclera or conjunctiva 1104 and the sclera 1106 of the eye to allow for the cutting surface to enter the vitreous humor 1108 section of the eye with the longitudinally movable shield protecting the cutting blade portion 900 during trocar insertion, then retracting the longitudinally movable shield after trocar insertion to expose the cutting portion 900.

Protecting the blade during trocar insertion prevents damage to the cutting blade, and/or the sleeve, during trocar insertion which would interfere with the ability of the depth-defined blade insertion were the sleeve to be perturbed. Thus, the longitudinally movable shield which can selectively protect the blade offers the ability to maintain the integrity of the sleeve, thereby preserving the ability of the vitreoretinal surgical instrument to penetrate a pre-selected depth of a target tissue.

In some embodiments, this disclosure includes the following numbered paragraphs:

A1. A vitreoretinal surgical instrument, comprising: a handheld small diameter blade extending from a base unit, having a length and a diameter equal to or smaller than 20 gauge, a support sleeve located around an end of the vitrectomy instrument portion adjacent to the base unit, and extending from the base unit to a location along the length of the vitrectomy instrument portion, wherein a distal end of the vitrectomy instrument portion is configured to enter an eye, while the support sleeve is configured to remain outside a surface of an eye when in use, wherein the support sleeve includes a rod.

A2. The vitrectomy instrument assembly of paragraph A1, wherein the small diameter vitrectomy instrument portion includes a 23, 25 or 27 gauge instrument.

A3. The vitrectomy instrument assembly of paragraph A1, wherein the support sleeve includes a stainless steel or titanium rod. In some aspects, the support sleeve comprises a material having superplasticity. In some aspects, the material is a shape memory alloy. In some aspects, the shape memory alloy is nitinol.

A4. The vitrectomy instrument assembly of paragraph A1, wherein the end of the rod tapers to a sharp blade structure.

A5. The vitrectomy instrument assembly of paragraph A1, wherein the tip of the blade is angled at 35 to 90 degrees, preferably about 45 degrees, to be configured in a “hockey stick” form.

A6. The shaft of the instrument can also be angled between 0 and 15 degrees to enable an improved angle of approach to the retina.

A7. The vitrectomy instrument assembly of paragraph A1, wherein the tip of the blade is exposed by varying amounts, usually from 0.5 to 2 mm and the rest of the blade is covered by a semi-rigid polymer guard depending on the type of membranes that needed to be peeled or dissected.

A8. The polymer guard in paragraph A6, wherein the tip of the guard is made of heat-shrinking polymer, and the blade inserts into the polymer guard and can be further secured to the blade with medical grade epoxy.

A9. The polymer guard in paragraph A6, wherein the polymer material can be comprised of, but not limited to, medical grade fluoroelastomer, polyolefin, polyvinylidene fluoride, silicone, silicone rubber, polyethylene, polyester, polyester block amide (PEBA) and other materials suitable for heat shrink tubing.

A10. The polymer guard in paragraph A6, wherein the tip of the guard fitted to the blade by applying heat to shrink down the polymer tubing to create a tight fit.

All. The polymer guard in paragraph A6, wherein the thickness of the polymer guard ranges from 0.1-1 mm.

The preceding merely illustrates the principles of various embodiments of the disclosure. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

All numbers expressing quantities or parameters used in the specification are to be understood as additionally being modified in all instances by the term “about”. Notwithstanding that the numerical ranges and parameters set forth, the broad scope of the patient matter presented herein are approximations, the numerical values set forth are indicated as precisely as possible. For example, any numerical value may inherently contain certain errors, evidenced by the standard deviation associated with their respective measurement techniques, or round-off errors and inaccuracies.

What is claimed is:

1. A vitreoretinal surgical instrument comprising:
a blade having a diameter greater than about 34 gauge comprising:
a cutting surface positioned at a first end of the blade and configured to cut an eye tissue;
a sleeve positioned proximate at least a portion of the blade and configured to displace at least some tissue proximate a predetermined cut location during use and further to control a depth to which the cutting surface of the blade enters tissue at the predetermined cut location during use and wherein the sleeve is positioned such that the sleeve substantially remains outside the surface of the eye during use;
a shaft coupled to blade; and
wherein the sleeve surrounds at least a portion of the blade.

2. The vitreoretinal surgical instrument of claim 1, wherein the diameter of the blade at a first end of the blade comprises greater than about 27 gauge, greater than about 25 gauge, or greater than about 23 gauge.

3. The vitreoretinal surgical instrument of claim 1, wherein the shaft comprises a stainless steel or titanium rod.

4. The vitreoretinal surgical instrument of claim 3, wherein an end of the shaft tapers to the blade.

5. The vitreoretinal surgical instrument of claim 1, wherein the blade comprises a tip section and a proximate section positioned such that an angle between the tip portion and the proximate section is in a range from about 45 degrees to about 135 degrees.

6. The vitreoretinal surgical instrument of claim 5, wherein the angle is about 90 degrees.

7. The vitreoretinal surgical instrument of claim 5, wherein the blade comprises a shape-memory metal.

8. The vitreoretinal surgical instrument of claim 7, wherein the blade can be configured to have a first angle and a second angle, wherein the first angle is present when the blade is deformed and the second angle is present when the blade is not deformed.

9. The vitreoretinal surgical instrument of claim 1, wherein the blade comprises a proximate section; and wherein the proximate section and the shaft are angled in a range from about 0 degrees to about 15 degrees to enable an improved angle of approach to the retina.

10. The vitreoretinal surgical instrument of claim 1, wherein blade comprises a tip section having an exposed section that has a length in a range from about 0.5 mm to about 2 mm; and wherein the sleeve comprises a semi-rigid polymer selected based on a type of membrane to be cut, peeled, or dissected.

11. The vitreoretinal surgical instrument of claim 1 wherein the sleeve comprises a polymer guard comprising heat-shrinking polymer, wherein the blade is surrounded at least in part by the polymer guard, and wherein the blade may be further secured to the polymer guard with medical grade epoxy.

12. The vitreoretinal surgical instrument of claim 1, wherein the sleeve comprises a polymer material comprising at least one of a medical grade fluoroelastomer, polyolefin, polyvinylidene fluoride, silicone, silicone rubber, polyethylene, polyester, polyester block amide (PEBA) and other materials suitable for heat shrink tubing.

13. The vitreoretinal surgical instrument of claim 1, wherein the sleeve comprises:
a guard having a tip fitted to the blade and polymer material;
wherein the tip is fitted by applying heat to shrink the polymer material to form a tight fit.

14. The vitreoretinal surgical instrument of claim 1, wherein the thickness of the sleeve is in a range from about 0.1 mm to about 1 mm.

15. The vitreoretinal surgical instrument of claim 1, wherein the sleeve comprises a drug.

16. The vitreoretinal surgical instrument of claim 1, wherein the drug is impregnated within the sleeve.

17. The vitreoretinal surgical instrument of claim 15, wherein the drug is a wound-healing drug, an antimicrobial drug, antibiotic, anesthetic, or combinations thereof.

18. The vitreoretinal surgical instrument of claim 17, wherein the wound-healing drug is an anti-inflammatory drug or an anti-VEGF drug.

19. The vitreoretinal surgical instrument of claim 15, wherein the drug is selected from: bevacizumab (Avastin), ranibizumab (Lucentis), aflibercept (Eylea), Vabysmo (faricimab), Ranibizumab-nuna (Byooviz), Ranibizumab-eqrn (Cimerli), Brolucizumab (Beovu), Faricimab (Vabysmo), Aflibercept (Eylea HD), Pegcetacoplan (Syfovre), Avacincaptad pegol (Izervay), Triamcinolone acetonide, Ganciclovir, Clindamycin, Foscarnet, Fomivirsen Methotrexate, Vancomycin, Ceftazidime, Amikacin, Amphotericin B, Voriconazole, and Dexamethasone.

20. A vitreoretinal surgical instrument assembly comprising:
a blade having a diameter greater than about 34 gauge comprising:
a material selected for a predetermined characteristic;
a cutting surface positioned at a first end of the blade and configured to cut tissue;
a sleeve positioned proximate at least a portion of the blade comprising:
a polymer selected for a predetermined property;
a thickness of the sleeve selected based on a type of tissue to be cut and/or dissected; and
wherein the sleeve is configured to displace at least some tissue proximate a predetermined cut location during, wherein the sleeve controls a depth to which the cutting surface of the blade enters the tissue at the predetermined cut location during use, and wherein the polymer selected for the sleeve allows the sleeve to substantially remain at or above a surface of the eye during use; and
a shaft coupled to blade; and
wherein the sleeve surrounds at least a portion of the blade.

21. The vitreoretinal surgical instrument of claim 20, wherein the polymer selected for a predetermined property is selected from medical grade fluoropolymer, fluoroelastomer, polyolefin, polyvinylidene fluoride, silicone, silicone rubber, polyethylene, polyester, and polyester block amide (PEBA).

22. The vitreoretinal surgical instrument of claim 20, wherein the material selected for a predetermined characteristic is selected from titanium, stainless steel, or nitinol.

23. The vitreoretinal surgical instrument of claim 20, wherein the sleeve comprises a thickness ranging from 0.1 mm to about 1 mm.

24. The vitreoretinal surgical instrument of claim 20, wherein the sleeve comprises a drug.

25. A method of dissecting tissue from an eye comprising:
positioning a vitreoretinal surgical instrument proximate a first tissue location,
wherein the vitreoretinal surgical instrument comprises:
a blade having a cutting surface;
a sleeve on the blade; and
applying a predetermined amount of pressure of the first tissue location to deflect an area of tissue to be cut and/or dissected during use.

26. The method of claim 25, wherein the vitreoretinal surgical instrument is a vitreoretinal surgical instrument of any of claim 1 or 20.

27. A surgical handpiece comprising:
a vitreoretinal surgical instrument of any of claim 1 or 20,
a longitudinally movable shield;
a rod-shaped body which includes a control slider;
wherein the control slider is configured to be movable along the longitudinal axis of the rod-shaped body;
wherein the control slider is configured to be positioned laterally on the rod-shaped body and is configured to control the longitudinal movement of the shield, and wherein the longitudinally movable shield is configured to have a first position where the shield is extended to cover the blade and a second position where the shield is retracted to expose the blade.

28. The surgical handpiece of claim 27, wherein the control slider comprises a protrusion configured to be manipulated by a digit of an operator.

29. The surgical handpiece of claim 27, wherein the longitudinally movable shield surrounds the blade when in the first position.

30. A kit comprising a surgical handpiece of any of claim 27 and a trocar.

31. A method of presenting a cutting blade to the vitreous humor of the eye of a subject, the method comprising:
inserting a trocar into the eye of a subject;
inserting a vitreoretinal surgical instrument comprising a blade through the trocar wherein the blade is surrounded by a longitudinally movable shield; and
retracting the longitudinally movable shield to expose the blade.
